SEQUENCE LISTING (1 sequence)

SEQ ID NO: 1 - protein, 431 aa, Artificial Sequence
  Aflibercept
SDTGRPFVEMYSEIPEIIHMTEGRELVIPCRVTSPNITVTLKKFPLDTLIPDGKRIIWDSRKGFIISNATYKEIGLLTCEATVNGHLYKTNYLTHRQTNTIIDVVLSPSHGIELSVGEKLVLNCTARTELNVGIDFNWEYPSSKHQHKKLVNRDLKTQSGSEMKKFLSTLTIDGVTRSDQGLYTCAASSGLMTKKNSTFVRVHEKDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

(19) United States
(12) Patent Application Publication
(10) Pub. No.: US 2020/0390693 A1
(43) Pub. Date: Dec. 17, 2020

(54) LIQUID COMPOSITION COMPRISING VEGF ANTAGONIST

(71) Applicant: SAMSUNG BIOEPIS CO., LTD., Incheon (KR)

(72) Inventors: Inae KIM, Seoul (KR); Youngseok JUNG, Incheon (KR); Yong Min JANG, Seoul (KR); Wonjung YOO, Busan (KR); Jaemin LEE, Seoul (KR); Yongkook KIM, Incheon (KR)

(21) Appl. No.: 16/770,858

(22) PCT Filed: Dec. 18, 2018
(86) PCT No.: PCT/KR2018/016156

§ 371 Date: Jun. 8, 2020

(30) Foreign Application Priority Data
Dec. 22, 2017 (KR) 10-2017-0178693

(51) Int. Cl. A61K 9/00; C07K 14/49; A61K 47/02; A61K 9/08; A61K 47/26

(52) U.S. Cl. CPC A61K 9/0048; C07K 14/49; C07K 2319/30; A61K 9/08; A61K 47/26; A61K 47/02

(57) ABSTRACT

Provided are: a composition for stabilizing a protein, the composition containing a buffer that includes phosphate, histidine, or a combination thereof, and a stabilizer that does not include sodium chloride and/or includes one or more selected form the group consisting of trehalose, sucrose and mannitol; a stabilized liquid composition of protein, containing the composition for stabilization and a protein; and a method for preparing a stabilized liquid composition by using the composition for stabilization.

### FIELD

Provided is a stabilized liquid formulation of a protein, and it relates to a composition for stabilization of a protein, comprising a buffer comprising phosphate, histidine, or a combination thereof, and a stabilizer, wherein the stabilizer comprises at least one selected from the group consisting of trehalose, sucrose and mannitol and/or does not comprise sodium chloride; a stabilized liquid composition comprising the composition for stabilization and a protein; and a preparation method of a stabilized liquid composition using the composition for stabilization.

### BACKGROUND

Fusion protein drugs are more likely to cause physicochemical instability due to their larger molecular weight and complex structure than general protein drugs, and therefore the development of a suitable formulation is required. The chemical and physical stability of protein drugs can be optimized by various conditions such as pH condition, buffer selection, a concentration of protein, an excipient and temperature, etc. For development of a formulation which can ensure the stability of fusion proteins and maintain pharmacological activity of the fusion proteins, various means such as buffer modification, pH optimization and stabilizer addition, and the like have been used.

EYLEA® (aflibercept), a macular degeneration therapeutic agent, is a liquid formulation drug which has a composition of 40 mg/mL aflibercept, pH 6.2, 10 mM sodium phosphate, 40 mM NaCl, 5% (w/v) sucrose, and 0.03% (w/v) polysorbate 20. In the case of intraocular drugs, the regulatory standards for sub-visible particles are more stringent than those for other general biopharmaceuticals (see Table 1), and thus it is needed to develop a formulation considering the particulate aspect.

TABLE 1
Sub-visible particle detection standards

Number of particles

| Category | ≥10 um | ≥25 um |
|---|---|---|
| Small-volume | <6,000/container | <600/container |
| (≤100 mL) injections | | |
| Large-volume | <25/mL | <3/mL |
| (>100 mL) injections | | |
| Ophthalmic solution | <50/mL | <5/mL |

* Based on USP 788, 789, light obscuration test standards

### SUMMARY

### Technical Problem

Accordingly, the present disclosure provides a composition for stabilization, which enhances the stability of a protein drug, and a stable liquid composition of a protein drug comprising the same.

An embodiment provides a composition for stabilization of a protein drug, comprising:

a buffer comprising sodium phosphate, histidine, or a combination thereof, and

a stabilizer which does not comprise sodium chloride and/or comprises at least one selected from the group consisting of trehalose, sucrose, mannitol and combinations thereof.

Another embodiment provides a liquid composition, comprising a protein; a buffer comprising sodium phosphate, histidine, or a combination thereof; and a stabilizer which does not comprise sodium chloride and/or comprises at least one selected from the group consisting of trehalose, sucrose, mannitol, and combinations thereof.

For example, the liquid composition may comprise

(1) a protein of 5 mg/ml to 100 mg/ml;

(2) a buffer of pH 4 to 8; and

(3) a stabilizer,

wherein the buffer comprises sodium phosphate, histidine, or a combination thereof,

and the stabilizer does not comprise sodium chloride and/or comprises at least one selected from the group consisting of trehalose, sucrose and mannitol.

The liquid composition may further comprise a surfactant, for example, in an amount of 0.01 to 3% (w/v) based on the total composition.

Another embodiment provides a pharmaceutical composition comprising the liquid composition.

For example, for the liquid composition or pharmaceutical composition, the protein may be one having a molecular weight of 10 to 500 kDa, 10 to 400 kDa, 10 to 300 kDa, 10 to 200 kDa, or 10 to 150 kDa. In an embodiment, the protein may be a VEGF antagonist, and for example, may be at least one selected from the group consisting of aflibercept (having a molecular weight of about 97 to 115 kDa), bevacizumab (having a molecular weight of about 149 kDa), ranibizumab (having a molecular weight of about 48 kDa), and the like.

When the protein is a VEGF antagonist, for example, one or more selected from the group consisting of aflibercept, bevacizumab, ranibizumab, and the like, the pharmaceutical composition may be an ophthalmic composition, and particularly, may be a parenteral formulation for intravitreal administration.

The liquid composition or pharmaceutical composition may be for intravitreal administration.

Still another embodiment provides a method of stabilizing a protein or a method of preparing a stabilized aqueous liquid composition, the method comprising a step of mixing the protein with the composition for stabilization.

### Technical Solution

In the present specification, a composition for stabilization to enhance the stability of a protein, a stable liquid composition of a protein comprising the same, a method for preparation of a stable aqueous liquid composition comprising a protein, and a method for stabilization of a protein are provided.

An embodiment provides a composition for stabilization of a protein, comprising a buffer comprising sodium phosphate, histidine, or a combination thereof, and a stabilizer which does not comprise sodium chloride and/or comprises at least one selected from the group consisting of trehalose, sucrose, and mannitol.

Another embodiment provides a liquid composition, comprising

(1) a protein;

(2) a buffer comprising sodium phosphate, histidine or a combination thereof; and

(3) a stabilizer which does not comprise sodium chloride and/or comprises one or more selected from the group consisting of trehalose, sucrose, and mannitol.

The protein content in the liquid composition may be 5 mg/ml to 100 mg/ml, 5 mg/ml to 80 mg/ml, 5 mg/ml to 60 mg/ml, 5 mg/ml to 50 mg/ml, 10 mg/ml to 100 mg/ml, 10 mg/ml to 80 mg/ml, 10 mg/ml to 60 mg/ml, 10 mg/ml to 50 mg/ml, 20 mg/ml to 100 mg/ml, 20 mg/ml to 80 mg/ml, 20 mg/ml to 60 mg/ml, 20 mg/ml to 50 mg/ml, 30 mg/ml to 100 mg/ml, 30 mg/ml to 80 mg/ml, 30 mg/ml to 60 mg/ml, or 30 mg/ml to 50 mg/ml, and for example, may be 5 mg/ml, 10 mg/ml, 20 mg/ml, 30 mg/ml, 40 mg/ml, 50 mg/ml, 60 mg/ml, 70 mg/ml, 80 mg/ml, 90 mg/ml, or 100 mg/ml.

The buffer may be one of pH 4 to 8, for example, pH 5.2 to 7.5, pH 5.2 to 7.2, pH 5.2 to 7, pH 5.2 to 6.8, pH 5.2 to 6.6, pH 5.2 to 6.4, pH 5.2 to 6.2, pH 5.5 to 7.5, pH 5.5 to 7.2, pH 5.5 to 7, pH 5.5 to 6.8, pH 5.5 to 6.6, pH 5.5 to 6.4, pH 5.5 to 6.2, pH 5.8 to 7.5, pH 5.8 to 7.2, pH 5.8 to 7, pH 5.8 to 6.8, pH 5.8 to 6.6, pH 5.8 to 6.4, pH 5.8 to 6.2, or pH 6.2.

The buffer may comprise one or more selected from the group consisting of phosphoric acid, acetic acid, citric acid, succinic acid, pharmaceutically acceptable salts (for example, sodium salt, potassium salt, etc.) of the acids, and histidine. In an embodiment, the buffer may comprise sodium phosphate, histidine, or a combination thereof. The buffer may be comprised at the concentration of about 1 mM to about 50 mM, about 1 mM to about 40 mM, about 1 mM to about 30 mM, about 1 mM to about 20 mM, about 1 mM to about 15 mM, about 1 mM to about 12 mM, about 5 mM to about 50 mM, about 5 mM to about 40 mM, about 5 mM to about 30 mM, about 5 mM to about 20 mM, about 5 mM to about 15 mM, about 5 mM to about 12 mM, about 8 mM to about 50 mM, about 8 mM to about 40 mM, about 8 mM to about 30 mM, about 8 mM to about 20 mM, about 8 mM to about 15 mM, about 8 mM to about 12 mM, or 10 mM, based on the total liquid composition.

In an embodiment, the stabilizer may not comprise sodium chloride.

In other embodiment, the stabilizer may be one or more selected from the group consisting of trehalose, sucrose, mannitol, and combinations thereof. For example, the stabilizer may comprise one or more kinds selected from the group consisting of trehalose of 1 to 20% (w/v), 1 to 15% (w/v), 1 to 10% (w/v), 5 to 20% (w/v), 5 to 15% (w/v), 5 to 10% (w/v), 7.8 to 20% (w/v), 7.8 to 15% (w/v), 7.8 to 10% (w/v), 7.8 to 8.2% (w/v), or 8% (w/v); sucrose of 1 to 20% (w/v), 1 to 15% (w/v), 1 to 10% (w/v), 5 to 20% (w/v), 5 to 15% (w/v), 5 to 10% (w/v), 7.8 to 20% (w/v), 7.8 to 15% (w/v), 7.8 to 10% (w/v), 7.8 to 8.2% (w/v), or 8% (w/v); and mannitol of 0.5 to 10% (w/v), 0.5 to 7.5% (w/v), 0.5 to 5% (w/v), 1 to 10% (w/v), 1 to 7.5% (w/v), 1 to 5% (w/v), 3 to 10% (w/v), 3 to 7.5% (w/v), 3 to 5% (w/v), 4 to 5% (w/v), or 4.5% (w/v).

In a specific embodiment, when the liquid composition comprises sodium phosphate as a buffer, or comprises phosphate (for example, sodium phosphate) as a buffer and sucrose as a stabilizer, the liquid composition may not comprise sodium chloride.

The liquid composition may comprise the aforementioned contents of a protein, a buffer and a stabilizer, and a residual aqueous medium (for example, water (purified water), saline solution, injection water, etc.).

In one embodiment, the liquid composition may further comprise a surfactant, for example, in an amount of 0.001 to 3% (w/v), 0.001 to 2% (w/v), 0.001 to 1% (w/v), 0.001 to 0.5% (w/v), 0.001 to 0.1% (w/v), 0.001 to 0.05% (w/v), 0.01 to 3% (w/v), 0.01 to 2% (w/v), 0.01 to 1% (w/v), 0.01 to 0.5% (w/v), 0.01 to 0.1% (w/v), 0.01 to 0.05% (w/v), or 0.03% (w/v), based on the total composition. The surfactant may be selected from any pharmaceutically acceptable surfactants which can disperse the protein evenly in the liquid composition medium. The surfactant may be a non-ionic surfactant; for example, at least one selected from the group consisting of polysorbates (for example, polysorbate 20 (polyoxyethylene (20) sorbitan monolaurate), polysorbate 40 (polyoxyethylene (20) sorbitan monopalmitate), polysorbate 60 (polyoxyethylene (20) sorbitan monostearate), polysorbate 80 (polyoxyethylene (20) sorbitan monooleate), wherein the numerical value behind the polyoxyethylene, i.e., (20), means the total number of oxyethylene groups (—(CH₂CH₂O)—); poloxamer (PEO-PPO-PEO copolymer; PEO: poly(ethylene oxide), PPO: poly(propylene oxide)), polyethylene-polypropylene glycol, polyoxyethylene compounds (for example, polyoxyethylene-stearate, polyoxyethylene alkyl ether (alkyl: C₁-C₃₀), polyoxyethylene monolauryl ether, alkylphenyl polyoxyethylene copolymer (alkyl: C₁-C₃₀), etc.), sodium dodecyl sulphate (SDS), and the like. For example, the surfactant may be polysorbates (for example, polysorbate 20).

The liquid composition provided in the present specification may be isotonic with a living tissue. For example, the osmotic pressure of the liquid composition may be about 200 mOsm/kg to about 400 mOsm/kg, for example about 250 mOsm/kg to about 300 mOsm/kg. Such an osmotic pressure may be adjusted by the stabilizer.

The electrical conductivity of the liquid composition provided in the present specification, may be about 0.1 mS/cm or more, for example, about 0.1 mS/cm to about 10 mS/cm, about 0.1 mS/cm to about 7 mS/cm, about 1 mS/cm to about 10 mS/cm, about 1 mS/cm to about 7 mS/cm, about 2.5 mS/cm to about 10 mS/cm, about 2.5 mS/cm to about 7 mS/cm, about 5 mS/cm to about 10 mS/cm, or about 5 mS/cm to about 7 mS/cm.

In the liquid composition provided in the present specification, the protein may be a protein drug, for example, a protein (for example, fusion protein) having a molecular weight of 10 to 500 kDa, 10 to 400 kDa, 10 to 300 kDa, 10 to 200 kDa, or 10 to 150 kDa. In an embodiment, the protein may be a VEGF (vascular endothelial growth factor) antagonist, for example, a VEGF-specific fusion protein in which a VEGF binding site derived from an extracellular domain of human VEGF receptor 1 and VEGF receptor 2, and the Fc region of human IgG1 are fused. In a specific embodiment, the VEGF-specific fusion protein may be a protein in which a region comprising immunoglobulin-like(Ig) domain 2 of human VEGF receptor 1 (Flt1) and Ig domain 3 of human VEGF receptor 2 (Flt1 or Flt4) and the Fc region of human IgG1 are fused; for example, the VEGF-specific fusion protein may be aflibercept having the following amino acid sequence of SEQ ID NO: 1.

Aflibercept amino acid sequence
(SEQ ID NO: 1)
SDTGRPFVEM YSEIPEIIHM TEGRELVIPC RVTSPNITVT
LKKFPLDTLI PDGKRIIWDS RKGFIISNAT YKEIGLLTCE
ATVNGHLYKT NYLTHRQTNT IIDVVLSPSH GIELSVGEKL
VLNCTARTEL NVGIDFNWEY PSSKHQHKKL VNRDLKTQSG
SEMKKFLSTL TIDGVTRSDQ GLYTCAASSG LMTKKNSTFV
RVHEKDKTHT CPPCPAPELL GGPSVFLFPP KPKDTLMISR
TPEVTCVVVD VSHEDPEVKF NWYVDGVEVH NAKTKPREEQ
YNSTYRVVSV LTVLHQDWLN GKEYKCKVSN KALPAPIEKT
ISKAKGQPRE PQVYTLPPSR DELTKNQVSL TCLVKGFYPS
DIAVEWESNG QPENNYKTTP PVLDSDGSFF LYSKLTVDKS
RWQQGNVFSC SVMHEALHNH YTQKSLSLSP G

(Disulfide Bridge: 30-79; 124-185; 246-306; 352-410, Dimer: 211; 214)

The fusion protein may be recombinationally or synthetically produced.

The liquid composition provided in the present specification may be stably maintained at the high temperature of about 40° C. for 4 weeks or more.

The term “stability is excellent” or “stably maintained” may mean that physical, chemical and/or biological properties and/or the structure of a protein in a composition can be maintained during storage (for example, during storage, a low protein polymer formation rate, a low protein aggregation rate, a low protein degradation rate, and/or a low denaturation rate, etc.). Various analysis techniques to measure the stability of the protein are well known in the related technical field.

For example, the liquid composition provided in the present specification, when the protein (antibody) content is 40 mg/ml, the change (% HMW at fourth week of storage−% HMW at week 0 (beginning of storage)) of the protein polymer formation rate or aggregation rate (High Molecular Weight % (w/v); % HMW) measured during storage at 40° C. for 4 weeks with a conventional SEC (size exclusion chromatography) may be about less than 9, for example, about 8.8 or less or about 8.3 or less, but not limited thereto.

Another embodiment provides a pharmaceutical composition comprising the liquid composition. The pharmaceutical composition may further comprise pharmaceutically acceptable carriers, diluents, and/or excipients. The pharmaceutically acceptable carrier is that conventionally used, and may comprise one or more selected from the group consisting of lactose, dextrose, sucrose, sorbitol, mannitol, starch, acacia rubber, calcium phosphate, alginate, gelatin, calcium silicate, microcrystal cellulose, polyvinylpyrrolidone, cellulose, water (for example, purified water), saline solution, syrup, methyl cellulose, methylhydroxybenzoate, propylhydroxybenzoate, talc, magnesium stearic acid, mineral oil, and the like, but not limited thereto.

The liquid composition or pharmaceutical composition may be administered through an oral or parenteral route. In case of parenteral administration (for example, injection), it may be administered by intravenous administration, subcutaneous administration, intramuscular administration, intraperitoneal administration, endothelial administration, local administration, intranasal administration, intrapulmonary administration, intrectal administration, intratumoral administration, intravitreal administration, etc.

In a specific embodiment, the liquid composition or pharmaceutical composition may be an ophthalmic solution comprising a VEGF antagonist as described above, and in this case, may be a parenteral formulation to be administered into a vitreous humor of an eye.

Another embodiment provides a method of stabilizing a protein or a method of preparing a stabilized liquid composition, comprising a step of mixing a protein with the aforementioned composition for stabilization.

In a specific embodiment, provided is a method for stabilization of a protein or a method for preparation of a stabilized aqueous liquid composition, comprising a step of mixing

(1) a protein;

(2) a buffer comprising sodium phosphate, histidine, or a combination thereof;

(3) a stabilizer which does not comprise sodium chloride and/or comprises one or more selected from the group consisting of trehalose, sucrose and mannitol; and

(4) optionally, a surfactant

The specific description of kinds and contents of each component which is used for the method for stabilization of a protein or the method of preparation of a stabilized aqueous liquid composition is same as aforementioned.

### Advantageous Effects

The present invention can inhibit production of polymers and/or aggregates and production of fragments and/or denaturation into charged variants which can be occurred during storage of the protein, thereby maintaining pharmacological effects of a protein in an extended period of time, by providing a composition and liquid formulation for stabilization that allow a protein such as a fusion protein to stably maintain physical, chemical and/or biological efficacy for an extended period of time.

### BRIEF DESCRIPTION OF THE DRAWINGS

FIG. 1 is a graph showing the % HMW (High-molecular weight %) change rate (4% HMW) for 4 weeks during 4 weeks storage at 40° C. of test compositions measured in Example 2.

FIG. 2 is a graph showing the result of analyzing statistically significant factors using Minitab (Minitab, version 17), based on the A % HMW result for 4 weeks during 4 weeks storage at 40° C. of test compositions measured in Example 2.

FIG. 3 is a graph showing the result of analyzing statistically significant factors using Minitab, based on the A % Acidic result for 4 weeks during 4 weeks storage at 40° C. of test compositions measured in Example 3.

FIG. 4 is a graph showing the result of analyzing statistically significant factors using Minitab, based on the % RPA (Relative Potency Activity) change rate (A % RPA) result for 4 weeks during 4 weeks storage at 40° C. of test compositions measured in Example 4.

### DETAILED DESCRIPTION

Hereinafter, the present invention will be described in more detail through examples and test examples. However, these examples and test examples are intended to illustrate the present invention, and should not be construed as limiting the present invention.

### Example 1. Preparation of Liquid Composition

Using a fusion protein functioning as a VEFG antagonist, Aflibercept (a fusion protein of a VEGF binding site derived from extracellular domains of human VEGF receptors 1 and 2, and the Fc region of human IgG1; CAS Number: 862111-32-8; SEQ ID NO: 1) as a protein drug, liquid Compositions 1 to 14 of the protein drug were prepared as the compositions of the following Table 2:

TABLE 2

| Composition | Protein drug | | | | |
|---|---|---|---|---|---|
| No. | concentration | pH | Buffer | Stabilizer | Surfactant |
| 1-3^{a} | 40 mg/mL | 6.2 | 10 mM sodium | 5%(w/v) Sucrose, | 0.03%(w/v) PS20 |
| | | | phosphate | 40 mM NaCl | |
| 4 | 40 mg/mL | 6.2 | 10 mM sodium | 8%(w/v) Sucrose | 0.03%(w/v) PS20 |
| | | | phosphate | | |
| 5 | 40 mg/mL | 6.2 | 10 mM sodium | 4.5%(w/v) Mannitol | 0.03%(w/v) PS20 |
| | | | phosphate | | |
| 6 | 40 mg/mL | 6.2 | 10 mM sodium | 8%(w/v) Trehalose | 0.03%(w/v) PS20 |
| | | | phosphate | | |
| 7 | 40 mg/mL | 6.2 | 10 mM sodium | 4.5%(w/v) dextrose | 0.03%(w/v) PS20 |
| | | | phosphate | | |
| 8 | 40 mg/mL | 6.2 | 10 mM | 8%(w/v) Sucrose | 0.03%(w/v) PS20 |
| | | | histidine | | |
| 9 | 40 mg/mL | 6.2 | 10 mM | 4.5%(w/v) Mannitol | 0.03%(w/v) PS20 |
| | | | histidine | | |
| 10 | 40 mg/mL | 6.2 | 10 mM | 8%(w/v) Trehalose | 0.03%(w/v) PS20 |
| | | | histidine | | |
| 11 | 40 mg/mL | 6.2 | 10 mM | 4.5%(w/v) dextrose | 0.03%(w/v) PS20 |
| | | | histidine | | |
| 12-14^{b} | 40 mg/mL | 6.2 | 10 mM sodium | 5%(w/v) Sucrose, | 0.03%(w/v) PS20 |
| | | | phosphate | 40 mM NaCl | |

^{a}Control (EYLEA ® composition-applied formulation),
^{b}Control (EYLEA ®)

### Example 2. Measurement of HMW Content (%)

To test the stability of Compositions 1-14 prepared in Example 1, % HMW (% high molecular weight; based on weight), indicating an aggregation degree of the protein drug in the compositions was measured with SE-HPLC during storage for 4 weeks at the conditions of protein drug concentration 40 mg/mL, pH 6.2 and 40° C.

More specifically, a substance, which was detected earlier than the time when a monomer was detected, was defined as HMW, and % HMW was measured under the condition of 1.0 mL/min flow rate and 17 min injection time using HPLC (Waters 2695 separation module alliance) and column (Tosoh, TSK-gel G3000 SWXL) (% HMW=Area_{H}/Area_{TOTAL}×100).

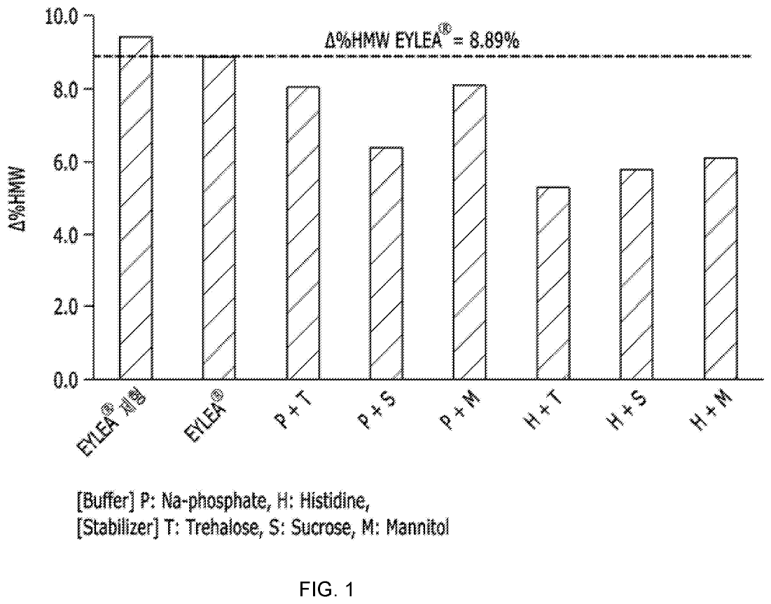

The measured % HMW (initial % HMW, 4^{th} week % HMW, Δ % HMW (4th week % HMW−initial % HMW) is showed in Table 3, and the 4^{th} week % HMW is shown in FIG. 1 (P: Na-phosphate, H: Histidine, T: Trehalose, S: Sucrose, M: Mannitol):

TABLE 3

| Composition | | | Initial | 40° C., 4^{th} week | |
|---|---|---|---|---|---|
| No. | Buffer | Stabilizer | % HMW | % HMW | Δ % HMW |
| 1-3^{a} | 10 mM | 5%(w/v) Sucrose, | 0.52 | 9.92 | 9.40 |
| (control) | Na-phosphate | 40 mM NaCl | [N = 3, SD: 0.02] | [N = 3, SD: 0.19] | [N = 3, SD: 0.21] |
| 4 | | 8%(w/v) | 0.52 | 6.92 | 6.40 |
| | | Sucrose | | | |
| 5 | | 4.5%(w/v) | 0.56 | 8.68 | 8.12 |
| | | Mannitol | | | |
| 6 | | 8%(w/v) | 0.55 | 8.62 | 8.07 |
| | | Trehalose | | | |
| 7 | | 4.5%(w/v) | 0.54 | 16.85 | 16.31 |
| | | Dextrose | | | |
| 8 | 10 mM Histidine | 8%(w/v) | 0.53 | 6.31 | 5.78 |
| | | Sucrose | | | |
| 9 | | 4.5%(w/v) | 0.54 | 6.64 | 6.10 |
| | | Mannitol | | | |
| 10 | | 8%(w/v) | 0.51 | 5.84 | 5.33 |
| | | Trehalose | | | |
| 11 | | 4.5%(w/v) | 0.53 | 13.19 | 12.66 |
| | | Dextrose | | | |

| 12-14^{b} | EYLEA ® | 1.46 | 10.35 | 8.89 |
|---|---|---|---|---|
| (control) | | [N = 3, SD: 0.00] | [N = 3, SD: 0.07] | [N = 3, SD: 0.07] |

^{a}Control (EYLEA ® composition-applied formulation)
^{b}Control (EYLEA ®)

As shown in Table 3 and FIG. 1, in all the test compositions comprising sucrose, trehalose or mannitol as a stabilizer, it was shown that both % HMW at the 4^{th} week and the rate of increase of % HMW at the 4^{th} week (compared to % HMW at the initial (0 week)) were lower than those of the control (EYLEA® and EYLEA® composition-applied formulation).

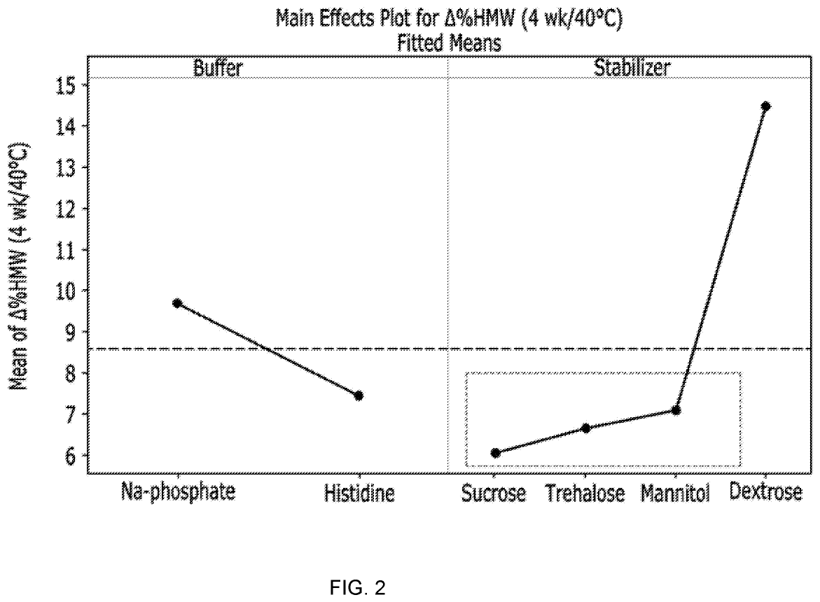

The result of analyzing statistically significant factors based on the Δ % HMW result shown in Table 3 is shown in FIG. 2. In FIG. 2, it was confirmed that the factors, which significantly affected Δ % HMW, were mannitol, sucrose, and trehalose, as a stabilizer. As shown in FIG. 2, there was no large difference depending on the kind of buffer, and in case of stabilizer, it was shown that % HMW was lower when using mannitol, sucrose or trehalose, compared to the composition using dextrose.

For the formulation comprising mannitol, sucrose, or trehalose as a stabilizer which was confirmed to lower Δ % HMW as above, the same test as above was repeatedly performed (n=3), and the obtained results are shown in Table 4:

TABLE 4

| Composition | | | Initial | 40° C., 4^{th} week | |
|---|---|---|---|---|---|
| No. | Buffer | Stabilizer | % HMW | % HMW | Δ % HMW |
| 1-3 | 10 mM Na-phosphate | 8% Trehalose | 0.78 | 8.56 | 7.78 |
| | | | [N = 3, SD: 0.01] | [N = 3, SD: 0.10] | [N = 3, SD: 0.09] |
| 4-6 | 10 mM Na-phosphate | 8% Sucrose | 0.80 | 8.07 | 7.27 |
| | | | [N = 3, SD: 0.01] | [N = 3, SD: 0.10] | [N = 3, SD: 0.09] |
| 7-9 | 10 mM Na-phosphate | 4.5% Mannitol | 0.78 | 9.58 | 8.80 |
| | | | [N = 3, SD: 0.01] | [N = 3, SD: 0.06] | [N = 3, SD: 0.06] |
| 10-12 | 10 mM Histidine | 8% Trehalose | 0.81 | 8.75 | 7.93 |
| | | | [N = 3, SD: 0.01] | [N = 3, SD: 0.15] | [N = 3, SD: 0.14] |
| 13-15 | 10 mM Histidine | 8% Sucrose | 0.78 | 8.41 | 7.63 |
| | | | [N = 3, SD: 0.00] | [N = 3, SD: 0.17] | [N = 3, SD: 0.17] |
| 16-18 | 10 mM Histidine | 4.5% Mannitol | 0.80 | 9.32 | 8.52 |
| | | | [N = 3, SD: 0.02] | [N = 3, SD: 0.12] | [N = 3, SD: 0.12] |
| 19-21 | 10 mM Na-phosphate | 5%(w/v) Sucrose, | 0.82 | 11.21 | 10.40 |
| | | 40 mM NaCl | [N = 3, SD: 0.00] | [N = 3, SD: 0.39] | [N = 3, SD: 0.391] |

As shown in Table 4, the formulation comprising mannitol, sucrose, or trehalose as a stabilizer showed lower Δ % HMW compared to the control, indicating that they have superior stability, and such superior stability was repeatedly achieved.

### Example 3. Measurement of Acidic Content (%) and Main Content (%)

Compositions 1-10 (Protein drug concentration: 40 mg/mL; pH 6.2; Surfactant: 0.03% (w/v) PS20) as shown in Table 5 were prepared using mannitol, sucrose, or trehalose as a stabilizer of which superior stabilizing effect was confirmed in Example 1 and Table 3, and using dextrose as a stabilizer for a control. To test the stability of these compositions, % Acidic (% Acidic variants content; based on weight) and % Main (content of a protein maintaining a surface charge of the initial condition in a charge variant aspect (based on weight)), which indicate the denaturation degree of the protein drug in the compositions, were measured using imaged capillary isoelectric focusing (icIEF) during storage for 4 weeks at 40° C.

More specifically, % Acidic, % Basic, and % Main were measured by analyzing acidic, main and basic isoforms of a sample, which was incubated after treating Enzyme (Sialidase A, Sigma-Aldrich), under the condition of 110 sec sample injection duration, 2000 psi sample injection pressure using icIEF instrument (Protein simple, iCE3).

The measured % Acidic and % Main (initial, 4^{th} week, 4^{th} week result—initial result (Δ % Acidic and Δ % Main)) are shown in Table 5:

TABLE 5

| | | | icIEF | | | | | |
|---|---|---|---|---|---|---|---|---|
| Composition | | | Initial | | 40° C., 4^{th} week | | | |
| No. | Buffer | Stabilizer | % Acidic | % Main | % Acidic | % Main | Δ % Acidic | Δ % Main |
| 1 | 10 mM | 8% Sucrose | 66.0 | 31.4 | 79.6 | 15.6 | 13.6 | −15.9 |
| 2 | Na-phosphate | 4.5% Mannitol | 64.9 | 30.4 | 78.8 | 15.6 | 13.9 | −14.8 |
| 3 | | 8% Trehalose | 65.4 | 31.1 | 78.2 | 15.6 | 12.8 | −15.6 |
| 4 | | 4.5% Dextrose | 67.9 | 29.2 | N/A^{a} | | | |
| 5 | 10 mM | 5% Sucrose, | 65.7 | 31.5 | 76.7 | 16.3 | 11.0 | −15.2 |
| | Histidine | 40 mM NaCl | | | | | | |
| 6 | | 8% Sucrose | 65.7 | 31.8 | 79.4 | 15.8 | 13.7 | −15.9 |
| 7 | | 4.5% Mannitol | 65.5 | 31.5 | 78.4 | 14.8 | 12.9 | −16.7 |
| 8 | | 8% Trehalose | 65.4 | 31.5 | 77.1 | 16.1 | 11.7 | −15.4 |
| 9 | | 135 mM NaCl | 65.9 | 31.2 | 78.1 | 15.8 | 12.2 | −15.4 |

| | | | | |
|---|---|---|---|---|
| 10 | 4.5% Dextrose | 66.7 | 28.9 | N/A^{a} |

^{a}N/A: Not available (Acidic, Main, and Basic peak were not distinguished)

As shown in Table 5, all of the tested compositions showed lower change degrees in both % Acidic and % Main compared to the comparative composition comprising dextrose as the stabilizer, which indicates that the test compositions have increased stabilities.

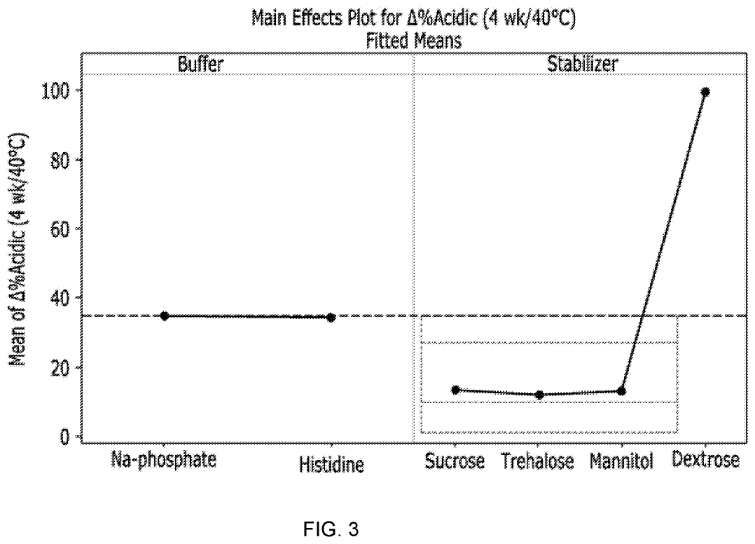

Statistically significant factors determined on the basis of the result of Table 5 were analyzed and the results are shown in FIG. 3. As shown in FIG. 3, it was confirmed that there was no significant difference of Δ % Acidic according to buffer kinds, but all the stabilizers except for dextrose significantly affected Δ % Acidic.

### Example 4. Measurement of VEGF Binding Efficacy of Aflibercept

To test the maintenance of activity of aflibercept which is a pharmacological active ingredient in Compositions 1-14 of Table 2 prepared in Example 1, % RBA (Relative Binding Activity) and % RPA (Relative Potency Activity) of aflibercept in the compositions to VEFG were measured during storage for 4 weeks at the condition of protein drug concentration 40 mg/mL, pH 6.2 and 40° C.

More specifically, % RBA was measured by the following method: after performing VEGF 165 coating (including washing and blocking; R&D system) on MaxiSorp 96 well plate (Nunc), Aflibercept and secondary antibody (Sigma) were loaded thereon in order, and then, TMB ELISA substance solution (Thermo Fisher Scientific) was treated; thereafter, % RBA (Relative Binding Activity) was measured using ELISA leader (SpectraMax).

The measured % RBA at 0 week and 4^{th} week at 40° C. and % RBA change rate for 4 weeks (Δ % RBA) are shown in Table 6:

TABLE 6

| | | | VEGF Binding | | |
|---|---|---|---|---|---|
| | | | (±3 SD, US: 84-113, EU: 86-114) | | |
| Composition | | | Initial | 40° C., 4^{th} week | |
| No. | Buffer | Stabilizer | % RBA^{C} | % RBA | % Δ RBA |
| 1-3^{a} | 10 mM | 5% Sucrose, | 101 | 90 | −11 |
| | Na-phosphate | 40 mM NaCl | [N = 3, SD: 4] | [N = 3, SD: 4] | [N = 3, SD: 7] |
| 4 | | 8% Sucrose | 95 | 82 | −13 |
| 5 | | 4.5% Mannitol | 97 | 101 | 4 |
| 6 | | 8% Trehalose | 99 | 94 | −5 |
| 7 | | 4.5% Dextrose | 99 | 70 | −29 |
| 8 | 10 mM | 8% Sucrose | 96 | 101 | 5 |
| 9 | Histidine | 4.5% Mannitol | 98 | 92 | −6 |
| 10 | | 8% Trehalose | 96 | 87 | −9 |
| 11 | | 4.5% Dextrose | 100 | 78 | −22 |

| | | | | |
|---|---|---|---|---|
| 12-14^{b} | EYLEA ® | 100 | 90 | −10 |

| | | |
|---|---|---|
| [N = 3, SD: 1] | [N = 3, SD: 2] | [N = 3, SD: 1] |

^{a}Control (EYLEA ® composition-applied formulation),
^{b}Control (EYLEA ®),
^{c}RBA (Relative Binding Activity)

As shown in Table 6, it was shown that % RBA decrease rate was lower or equal level to that of the control in all the tested compositions, except for the composition comprising dextrose as the stabilizer, and such a result showed that aflibercept maintained the binding activity to VEGF well in all the tested compositions.

In addition, % RPA was measured by the following method: after loading and incubation of KDR 293 cell (Promega) in a 96 well plate in which aflibercept and VEGF were loaded in order, % RPA (Relative Potency Analysis) was analyzed using Envision microplate reader (Perkin Elmer, Envision 2014).

The measured % RPA at 0 week and 4th week at 40° C. and % RPA change rate for 4 weeks (Δ % RPA) are shown in Table 7:

TABLE 7

| | | | VEGF Neutralization | | |
|---|---|---|---|---|---|
| | | | (±3 SD, US: 83-118, EU: 92-111) | | |
| Composition | | | Initial | 40° C., 4^{th} week | |
| No. | Buffer | Stabilizer | % RPA^{C} | % RPA | Δ % RPA |
| 1-3^{a} | 10 mM | 5% Sucrose, | 103 | 91 | −8 |
| | Na-phosphate | 40 mM NaCl | [N = 3, SD: 5] | [N = 3, SD: 5] | [N = 3, SD: 3] |
| 4 | | 8% Sucrose | 96 | 86 | −10 |
| 5 | | 4.5% Mannitol | 98 | 86 | −12 |
| 6 | | 8% Trehalose | 100 | 83 | −17 |
| 7 | | 4.5% Dextrose | 109 | 77 | −32 |
| 8 | 10 mM | 8% Sucrose | 102 | 97 | −5 |
| 9 | Histidine | 4.5% Mannitol | 97 | 97 | 0 |
| 10 | | 8% Trehalose | 98 | 95 | −3 |
| 11 | | 4.5% Dextrose | 109 | 89 | −20 |

| | | | | |
|---|---|---|---|---|
| 12-14^{b} | EYLEA ® | 105 | 95 | −11 |

| | | |
|---|---|---|
| [N = 3, SD: 7] | [N = 3, SD: 1] | [N = 3, SD: 6] |

^{a}Control (EYLEA ® composition-applied formulation),
^{b}Control (EYLEA ®),
^{c}RPA (Relative Potency Activity)

As shown in Table 7, except for the composition comprising dextrose as the stabilizer, the decrease rates in % RPA of all the tested compositions were lower or equal level to that of control, which indicates that the relative titre of aflibercept to VEGF neutralization capability can be maintained well in all the tested compositions.

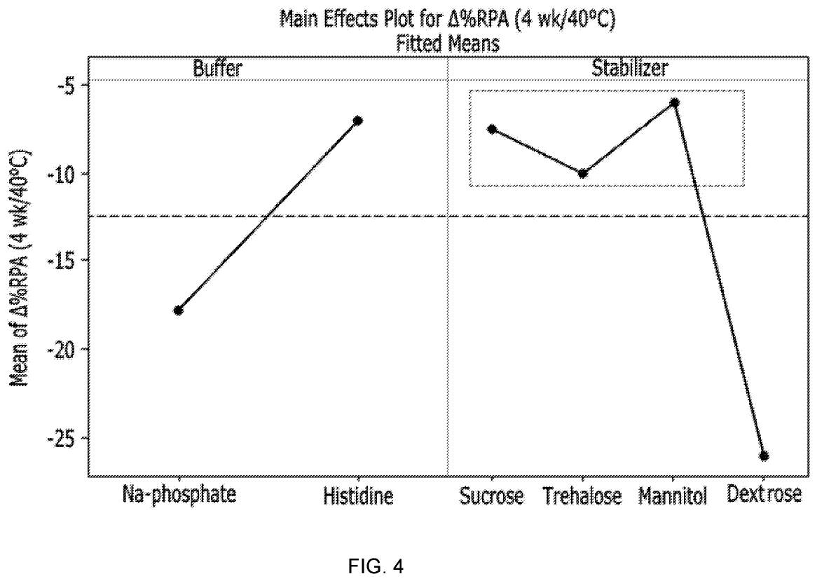

Statistically significant factors were analyzed based on the % RPA result in Table 7 and the obtained results are shown in FIG. 4. As shown in FIG. 4, all the tested buffers showed similar or improved % RPA decrease, compared to the control, and in case of stabilizer, all the tested stabilizers except for dextrose showed similar or less % RPA decrease compared to the control.

### Example 5. Measurement of Stability of 6 Kinds of Compositions

% Main of 6 kinds of compositions (compositions 4, 5, 6, 8, 9 and 10 in Table 2) which were confirmed to have excellent stability in Examples 2-4, among compositions prepared in Example 1, was measured using CE-SDS (Capillary electrophoresis sodium dodecyl sulfate) (40° C., 4 weeks).

More specifically, % Main was measured by the following method: after thermal treatment at 70° C. of a sample which is mixed with SDS sample buffer (AB Sciex) and BME (2-mercaptoethaol, Sigma Aldrich) (mixing ratio: about 1:50 (v:v)), % total protein area which was detected under 220 nm was analyzed, using CE analysis instrument (Beckman Coulter, Pa. 800 plus) and 32 karat software (Beckman Coulter).

The measured % Main at 0 week and 4th week at 40° C. and % Main change rate for 4 weeks (Δ % Main) are shown in Table 8:

TABLE 8

| Composition | | | Initial | | 40° C., 4^{th} week | |
|---|---|---|---|---|---|---|
| No. | Buffer | Stabilizer | % Impurity | % Main | % Main | Δ % Main |
| 1-3^{a} | 10 mM | 5%(w/v) Sucrose, | 3.1 | 96.9 | 95.2 | −1.7 |
| | Na-phosphate | 40 mM NaCl | [N = 3, SD: 0.37] | [N = 3, SD: 0.37] | [N = 3, SD: 0.12] | [N = 3, SD: 0.24] |
| 4 | | 8%(w/v) Sucrose | 2.7 | 97.3 | 96.3 | −1.0 |
| 5 | | 4.5%(w/v) Mannitol | 3.1 | 96.9 | 95.9 | −1.0 |
| 6 | | 8%(w/v) Trehalose | 2.6 | 97.4 | 95.3 | −2.1 |
| 8 | 10 mM | 8%(w/v) Sucrose | 2.0 | 98.0 | 96.3 | −1.7 |
| 9 | Histidine | 4.5%(w/v) Mannitol | 2.9 | 97.0 | 96.6 | −0.4 |
| 10 | | 8%(w/v) Trehalose | 2.1 | 97.8 | 96.3 | −1.5 |

^{a}Control (EYLEA ® composition-applied formulation)

As shown in Table 8, the % Main decrease rate in tested 6 compositions were equivalent to or lower than that of the control. Such a result showed that all the tested 6 compositions possessed equivalent to or higher stability compared to the control.

1-28. (canceled)

29. A liquid composition comprising:
(1) a VEGF antagonist of 5 mg/ml to 100 mg/ml;
(2) a buffer of 1 to 50 mM, comprising phosphoric acid or a pharmaceutically acceptable salt of the acid and having pH ranging from 4 to 8; and
(3) a stabilizer of 7.8 to 10% (w/v),
wherein the liquid composition does not comprise sodium chloride.

30. The liquid composition according to claim 29, wherein the amount of the VEGF antagonist is 20 mg/ml to 60 mg/ml.

31. The liquid composition according to claim 29, wherein the pH of the liquid composition is 5.8 to 6.2.

32. The liquid composition according to claim 29, wherein the concentration of the buffer in the composition is 1 to 20 mM.

33. The liquid composition according to claim 29, wherein the buffer is sodium phosphate.

34. The liquid composition according to claim 29, wherein the amount of the stabilizer is 7.8 to 8.2% (w/v).

35. The liquid composition according to claim 29, wherein the stabilizer is at least one selected from the group consisting of trehalose, sucrose, and a combination thereof.

36. The liquid composition according to claim 29, further comprising a surfactant.

37. The liquid composition according to claim 36, wherein the surfactant is comprised in an amount of 0.001 to 3% (w/v), based on the total composition.

38. The liquid composition according to claim 36, wherein the surfactant is at least one selected from the group consisting of polysorbate 20, polysorbate 80, and a combination thereof.

39. The liquid composition according to claim 29, which is aqueous and isotonic.

40. The liquid composition according to claim 29, wherein the composition is aqueous and has osmotic pressure of 200 to 400 mOsm/kg.

41. The liquid composition according to claim 29, wherein the VEGF antagonist is aflibercept.

42. A parenteral formulation for intravitreal administration, comprising the liquid composition according to claim 29.

43. A liquid composition comprising,
(1) aflibercept of 30 mg/ml to 50 mg/ml;
(2) a buffer of 1 to 20 mM, comprising sodium phosphate and having pH ranging from 5.2 to 7.2;
(3) a stabilizer of 7.8 to 10% (w/v); and
(4) a surfactant of 0.01 to 0.1% (w/v),
wherein the liquid composition does not comprise sodium chloride.

44. The liquid composition according to claim 43, wherein the pH of the liquid composition is 5.8 to 6.2.

45. The liquid composition according to claim 43, wherein the amount of the stabilizer is 7.8 to 8.2% (w/v).

46. The liquid composition according to claim 43, wherein the stabilizer is at least one selected from the group consisting of trehalose, sucrose, and a combination thereof.

47. The liquid composition according to claim 43, wherein the surfactant is at least one selected from the group consisting of polysorbate 20, polysorbate 80, and a combination thereof.

48. A parenteral formulation for intravitreal administration, comprising the liquid composition according to claim 43.

49. A liquid composition comprising,
(1) aflibercept of 30 mg/ml to 50 mg/ml;
(2) a buffer of 1 to 20 mM, comprising sodium phosphate;
(3) a sucrose of 7.8 to 10% (w/v); and
(4) a polysorbate 20 of 0.01 to 0.1% (w/v);
wherein the liquid composition has pH of 6.2 and does not comprise sodium chloride.
